(19) United States
(12) Patent Application Publication
(10) Pub. No.: US 2005/0232892 A1
(43) Pub. Date: Oct. 20, 2005

(54) Hair conditioning composition

(76) Inventors: Edward Banks, Wirral (GB); Collin Christopher David Giles, Jatujak (TH)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/518,561

(22) PCT Filed: Jun. 12, 2003
(86) PCT No.: PCT/EP03/05211

§ 371 Date: May 27, 2005

(30) Foreign Application Priority Data
Jun. 21, 2002 (EP) 02254336.7
Dec. 11, 2002 (GB) 0228878.5

(51) Int. Cl. A61K 7/075; A61K 7/08

(52) U.S. Cl. 424/70.27

(57) ABSTRACT

A hair conditioning composition with a pH of 8 or less comprises:(a) 0.01% to 10% by weight of one or more cationic surfactants; (b) 0.01% to 10% by weight of one or more fatty alcohols having from 18 to 22 carbon atoms; (c) 0.001% to 5% by weight of a hydrophobically modified clay; and (d) water wherein, the weight ratio of cationic surfactant to fatty alcohol is from 1:1 to 1:10. The compositions can impart improved conditioning benefits to hair.

This invention relates to a hair conditioning composition, to a method of treating hair using the composition and to the use of certain materials for imparting conditioning benefits to hair.

Hair conditioning compositions have been available for several years. Hair conditioning compositions are typically intended for use after the hair has been washed and can impart properties to the hair such as smoothness, shine, ease of combing, improved style retention and improved hold. The compositions are generally applied to clean hair whilst it is still wet after having been shampooed, and are then rinsed off.

Hair conditioning compositions constitute a significant proportion of the market for hair treatment products but not all of the existing products provide the optimum balance of the individual properties that make up conditioning benefits. Therefore, there remains a need for compositions that can impart improved conditioning benefits to hair and/or an altered profile of the properties that make up what are perceived by the user of the compositions as hair conditioning benefits. For example, there can be an advantage in increasing the perceived smoothness of hair which can be provided by a conditioning composition compared to other hair conditioning properties.

Certain hydrophobically modified clays have been incorporated into hair treatment compositions as suspending or anti-agglomerating agents.

For example, U.S. Pat. No. 4,983,383 discloses hair care compositions which are said to give both improved style and hair conditioning properties. Hydrophobically modified clays are described as being suitable suspending/antiagglomerating agents for silicone gums in hairspray forms of the compositions disclosed. The hydrophobically modified clays are, accordingly, employed in the examples of hairspray compositions that are given in the document. However, there is no suggestion of using these materials in compositions other than hairsprays or of any other benefit that the clays might possess.

U.S. Pat. No. 5,846,549 discloses cosmetic compositions intended for cleaning the hair, the scalp and/or the skin. The compositions employ the clay as an agent for dispersing an insoluble silicone in the composition. Generally, the clay is not modified. The compositions are cleaning compositions, such as shampoos, and there is no mention of hair conditioning compositions.

JP-A-05/246,824 discloses hair cosmetics which are water-in-oil emulsions. The cosmetics contain an organically modified clay material, a quaternary ammonium salt, a water-soluble high molecular weight substance and a silicone. The cosmetics are stated as being non-sticky and as imparting lustre, smooth feel and firmness.

JP-A-61/066,752 describes a gel composition containing a silicone oil, a polyoxyethylene-modified silicone and an organically modified montmorillonite clay. The compositions aim to have good stability. There is no mention of rinse off hair treatment compositions.

U.S. Pat. No. 5,679,327 describes highly alkaline hair straightening emulsions. Examples cited in the disclosure include a modified hectorite clay gellant with cationic surfactant and high levels of cetearyl alcohol.

WO 99/25312 discloses fluidized polymer suspensions. An example in the disclosure contains Tixogel MP 100 clay as a suspending agent along with cationic surfactant and cetyl alcohol. The weight ratio of cationic surfactant to fatty alcohol is 1:0.67.

The present invention aims to provide hair conditioning compositions that can impart improved conditioning benefits to hair and/or an altered profile of the individual properties that make up conditioning benefits.

According to the present invention in a first aspect, there is provided a hair conditioning composition with a pH of 8 or less comprising:
(a) 0.01% to 10% by weight of one or more cationic surfactants;
(b) 0.01% to 10% by weight of one or more fatty alcohols having from 8 to 22 carbon atoms;
(c) 0.01% to 5% by weight of a hydrophobically modified clay; and
(d) water,
wherein the weight ratio of cationic surfactant to fatty alcohol is from 1:1 to 1:10.

In another aspect, the present invention provides a method of treating hair which comprises applying to the hair a composition of the invention.

A further aspect of the invention is the use of a hydrophobically modified clay for imparting conditioning benefits to hair.

The present invention is based on the surprising finding that a hydrophobically modified clay can provide hair conditioning benefits in certain hair conditioning compositions. Hair conditioning benefits that can be provided by the compositions of the invention can be imparted to wet and/or dry hair and include one or more of smoothness, shine, ease of combing, style retention, hold, volume, coated feel, bounce, lift, clean feeling, hold, reduced flyaway, reduced dryness, coated feel, fineness, moisturised and powdery but clean. These one or more properties can be improved compared to otherwise identical compositions that do not contain the hydrophobically modified clay.

The hair conditioning compositions of the invention are rinse off compositions ie, they are intended to be rinsed from the hair (typically with water) after use. Such rinse off compositions are distinguished from leave on compositions that are intended not to be rinsed off the hair but instead left on the hair, until after the hair is dry.

The hair conditioning compositions of the invention are typically aqueous-based compositions which comprise an aqueous phase, optionally comprising a gelled phase and/or an insoluble oil phase dispersed and/or suspended in the aqueous phase. As such, the compositions are not water-in-oil emulsions.

Compositions according to the invention have a pH of 8 or less, preferably 7 or less, more preferably 6.5 or less. Suitably, the pH is 2.5 or more, preferably 4 or more.

The compositions of the invention comprise a hydrophobically modified clay in an amount of from 0.01% to 5% by weight, preferably from 0.01% to 3% by weight, more preferably from 0.05% to 1% by weight based on the total weight of the composition. Higher levels of hydrophobically modified clays can give unpleasant tactile properties to the composition for some consumers.

Hydrophobically modified clays may be used in the present invention either singly or in combination with one or more other hydrophobically modified clays.

Suitable clays include hydrophobically modified natural clays and synthetic clays. In general, the term clay refers to a composition comprising particles which have a net electrostatic (ie, positive or negative charge) on at least one surface.

Preferably, the hydrophobically modified clay has a layered structure. In the compositions of the invention, the hydrophobically modified clay is advantageously present in the form of a dispersion or suspension of the clay particles.

Hydrophobically modified clays of the invention may be anionic or cationic, ie, they may have a net charge on the surface of the clay that is negative or positive, respectively. The term anionic clays and related terms, as used herein, refers to clays which are themselves anionic in nature ie, the clays themselves are negatively charged at their surface and are capable of exchanging cations. Similarly, the term cationic clays and related terms, as used herein, refers to clays which are themselves cationic in nature ie, the clays themselves are positively charged at their surface and are capable of exchanging anions.

Hydrophobically modified clays are derivable from clays by modification of the clay with a hydrophobic material.

Preferred anionic clays are clays from the smectite class of clays. Typically, clays of this type are crystalline, expandable, three-layer clays.

Smectite clays are, for example, disclosed in U.S. Pat. Nos. 3,862,058, 3,948,790, 3,954,632 and 4,062,647 and in EP-A-299,575 and EP-A-313,146, all in the name of Procter & Gamble Company.

The term smectite clays herein includes both the clays in which aluminium oxide is present in a silicate lattice and the clays in which magnesium oxide is present in a silicate lattice. Typical smectite clay compounds include the compounds having the general formula Al₂(Si₂O₅)₂(OH)₂.nH₂O and the compounds having the general formula Mg₃(Si₂O₅)₂(OH)₂.nH₂O, and derivatives thereof, for example in which a proportion of the aluminium ions are replaced with magnesium ions or a proportion of the magnesium ions are replaced with lithium ions and/or some of the hydroxyl ions are replaced by fluoride ions; the derivatives may comprise a further metal ion to balance the overall charge. Smectite clays tend to adopt an expandable, three-layer structure.

The hydrophobically modified clay is preferably an expandable three-layer clay comprising at least 75% by weight of the clay of atoms selected from oxygen, silicon and aluminum and/or magnesium. More preferably, the hydrophobically modified clay comprises atoms selected from oxygen, silicon and aluminum and/or magnesium in an amount of at least 5% by weight of the clay, for each of the atoms.

Specific examples of suitable smectite clays include those selected from the classes of the montmorillonites, hectorites, volchonskoites, nontronites, saponites, beidelites and sauconites, particularly those having an alkali or alkaline earth metal ion within the crystal lattice structure. Particularly preferred are hectorites, montmorillonites, nontronites, saponites, beidelites, sauconites and mixtures thereof. Preferred are montmorillonites, eg, bentonites and hectorites, with bentonites being particularly preferred.

The hydrophobically modified clay is preferably a hydrophobically treated bentonite clay.

It is customary to measure cation exchange capacity (sometimes termed “base exchange capacity”) in terms of milliequivalents per 100 g of clay (meq/100 g). The cation exchange capacity of clays can be measured in several ways, including by electrodialysis, by exchange with ammonium ion followed by titration or by a methylene blue procedure, all as fully set fourth in Grimshaw, “The Chemistry and Physics of Clays”, pp. 264-265, Interscience (1971). The cation exchange capacity of a clay mineral relates to such factors as the expandable properties of the clay and the charge of the clay, which, in turn, is determined at least in part by the lattice structure, and the like.

Preferred anionic clays for use in the present invention have an ion exchange capacity of from 0.7 meq/100 g to 150 meq/100 g. Particularly preferred are clays having an ion exchange capacity of from 30 meq/100 g to 100 meq/100 g.

The clays preferably have an average particle size in the range of from 0.0001 μm to 800 μm, more preferably from 0.01 μm to 400 μm such as from 0.02 μm to 220 μm, even more preferably 0.02 μm to 100 μm. Particle sizes can be determined using a Malvern Mastersizer (Malvern Instruments, UK).

The hydrophobically modified clays used in the compositions of the invention preferably have organic ions replacing at least a proportion of inorganic metal ions by ion exchange processes known in the art. Preferably, the clay is hydrophobically modified by exchange into the clay of cations comprising one or more alkyl groups containing from 6 to 30 carbon atoms. The cationic group is preferably a quaternary ammonium group. Advantageously, the cations have the formula N⁺R¹R²R³R⁴ wherein R¹, R², R³ and R⁴ are independently (C₁ to C₃₀) alkyl, preferably (C₆ to C₃₀) alkyl, or benzyl. Preferably, one, two or three of R¹, R², R³ and R⁴ are independently (C₆ to C₃₀) alkyl and the other R¹, R², R³ and R⁴ group or groups are (C₁-C₆) alkyl or benzyl. Suitably, two of R¹, R², R³ and R⁴ are independently (C₆ to C₃₀) alkyl and the other R¹, R², R³ and R⁴ groups are (C₁-C₆) alkyl or benzyl. Optionally, the alkyl groups may comprise one or more ester (—OCO— or —COO—) and/or ether (—O—) linkages within the alkyl chain. Alkyl groups may be straight chain or branched and, for alkyl groups having 3 or more carbon atoms, cyclic. The alkyl groups may be saturated or may contain one or more carbon-carbon double bonds (eg, oleyl). The alkyl groups are optionally substituted with one or more hydroxyl groups. Alkyl groups are optionally ethoxylated with one or more ethyleneoxy groups on the alkyl chain. Preferably, the alkyl groups are straight chain, saturated groups.

Preferred compounds of formula N⁺R¹R²R³R⁴ which have two (C₆ to C₃₀) alkyl groups include:
Distearyldimethylammonium chloride (distearyl dimonium chloride);
Distearyldimethylammonium bromide (distearyl dimonium bromide);
Dicetyldimethylammonium chloride (dicetyl dimonium chloride);
Dicetyldimethylammonium bromide (dicetyl dimonium bromide);
Dimethyldi (hydrogenated tallow)ammonium chloride (Quaternium-18);
Dicetylmethylbenzylammonium chloride;
Dicocodimethylammonium chloride (dicoco dimonium chloride);
Dicocodimethylammonium bromide (dicoco dimonium bromide);
Dibehenyl/diarachidyldimethylammonium bromide (dibehenyl/diarachidyl dimonium bromide);
Dibehenyl/diarachidyldimethylammonium chloride (dibehenyl/diarachidyl dimonium chloride);
Dibehenyl dimonium methyl sulfate (dibehenyl dimonium methyl sulfate);
Hydroxypropyl bis-stearylammonium chloride (hydroxypropyl bis-stearyl dimonium chloride);
Dibehenyldimethylammonium chloride (dibehenyl dimonium chloride);
Dimyristyldimethylammonium chloride (dimyristyl dimonium chloride); and
Dimyristyldimethylammonium bromide (dimyristyl dimonium bromide).

Preferred compounds of formula N⁺R¹R²R³R⁴ which have three (C₆ to C₃₀) alkyl groups include compounds which have three alkyl groups having 8 to 22 carbon atoms and one alkyl group having 1 to 4 carbon atoms, such as, for example:
Tricetylmethylammonium chloride;
Tricetylmethylammonium bromide;
Tricetylmethylammonium methylsulfate;
Tri((C₈-C₁₀)alkyl)methylammonium chloride;
Tri((C₈-C₁₀)alkyl)methylammonium bromide; and
Tri((C₈-C₁₀)alkyl)methylammonium methylsulfate.

A particularly preferred material is bentonite modified with Quaternium-18 (ie, di-hydrogenated tallow dimethyl ammonium cations). An example of such a product is Tixogel MP 100™ from Sud Chemie. Other suitable hydrophobically modified clays include Quaternium benzalkonium bentonite, Quaternium-18 hectorite, stearalkonium bentonite, stearalkonium hectorite and dihydrogenated tallow benzylmonium hectorite.

In a preferred embodiment of the invention, the composition further comprises one or more hair fibre modifying agents that are capable of reducing the stiffness of a hair fibre. Hair fibre modifying agents have been found to give particular advantages in terms of the hair conditioning properties of the compositions when used in combination with hydrophobically modified clays according to the invention. Hair fibre modifying agents can be identified by the skilled person by measuring the change in stiffness of hair fibres using the method described herein in the examples section below.

Hair fibre modifying agents for use in the invention are preferably capable of reducing the stiffness of a hair fibre by at least 5%, more preferably at least 10%, even more preferably at least 15% relative to the stiffness of the original (ie, untreated) hair fibre.

Hair fibre modifying agents can be insoluble in water at 25° C. (ie, having a solubility of less than 1 gram per litre, preferably less than 0.1 gram per litre in water). Such hair fibre modifying agents will typically be liquid at 25° C. Examples of insoluble hair fibre modifying agents include the following (u denotes unified atomic mass units):
mineral oils, preferably those having a low viscosity and/or low molecular weight, typically a molecular weight below 600 u (eg, from 120 to 600 u) and/or a viscosity below 4 cSt (mm²sec⁻¹) as measured using a Brookfield viscometer at 18° C. using an LV1 spindle;

for example, oils comprising straight chain or branched chain, saturated or unsaturated hydrocarbons having from 10 to 44 carbon atoms, optionally comprising one or more phenyl groups;
functionalised oils, preferably compounds containing one or more groups selected from ether, ester, keto, aldehyde, carboxyl, alcohol, diol, polyol, amino, amido, thiol, thioether, and preferably containing 8 to 44 carbon atoms; specific examples are isoamyl ether, isopropyl myristate, octan-2-one, decyl alcohol and 1,10-decanediol;
saccharide polyesters (eg, esters of sucrose with carboxylic acids having from 4 to 36 carbon atoms, such as sucrose pentaerucate);
silicone oils, preferably low molecular weight (eg, having a molecular weight of below 2000 u such as from 300 to 2000 u) straight chain dimethicones or cyclomethicones; a suitable commercial product is DC 245 from Dow Corning;
triglyceride oils, preferably those containing acyl groups comprising from 6 to 24 carbon atoms (including the carbon atom of the C═O group).

A particularly preferred hair fibre modifying agent of the type mentioned above is a hydrocarbon oil. The hydrocarbon oils may comprise straight chain or branched alkanes having from 8 to 16 carbon atoms. Most preferred hydrocarbon oils are light mineral oils, such as paraffin oils eg, C11-C13 isoparaffin oils. An example of a commercial product is Isopar L™ which is available from Exxon.

Insoluble hair fibre modifying agents are typically hydrophobic. By the term hydrophobic in relation to the hair fibre modifying agents, it is meant that the agents have a greater solubility in octan-1-ol than water at 25° C. Preferably, the agents have a log P value (wherein P is the n-octanol/water partition coefficient and log P means log₁₀ P) of greater than 1, preferably greater than 2, more preferably greater than 3. Log P values can be determined as described in J Sangster, Octanol-water partition coefficients of simple organic compounds, J Phys Chem Ref Data, 18, 1111, 1989, the contents of which are incorporated by reference herein, and are typically determined at 25° C. Preferably, log P values are measured at 25° C.

Alternatively, hair fibre modifying agents for use in the invention can be soluble in water at 25° C. (ie, having a solubility of at least 1 gram per litre in water). Examples of soluble hair fibre modifying agents include the following:
amino acids (such as any of the twenty naturally occurring amino acids in optically active (eg, enantiomeric) or racemic forms), for example arginine and lysine;
urea;
methyl amines or their N-oxides, for example betaine and trimethylamine oxide;
mono-, di- or poly-carboxylic acids, preferably having from 1 to 12 carbon atoms and optionally comprising one or more hydroxyl groups, such as citric acid and lactic acid;
carbohydrates, such as mono- or di-saccharides eg, glucose and sucrose; and
polyols, such as sorbitol, mannitol and glycerol.

Urea is a particularly preferred water-soluble hair fibre modifying agent.

The one or more hair fibre modifying agents are preferably present in the composition in an amount of from 0.001% to 10% by weight, more preferably 0.01% to 5% by weight, most preferably 0.01% to 2% by weight.

Compositions in accordance with the invention are formulated as conditioners for the treatment of hair (typically after shampooing) and subsequent rinsing.

The compositions comprise one or more conditioning surfactants, which are cosmetically acceptable and suitable for topical application to the hair.

Suitable conditioning surfactants include those selected from cationic surfactants, used singly or in admixture. Examples include quaternary ammonium hydroxides or salts thereof, eg, chlorides, such as of the type mentioned hereinbefore. Preferably, the cationic surfactants have the formula N⁺R^{1a}R^{2a}R^{3a}R^{4a} wherein R^{1a}, R^{2a}, R^{3a} and R^{4a} are independently (C₁ to C₃₀) alkyl or benzyl. Preferably, one, two or three of R^{1a}, R^{2a}, R^{3a} and R^{4a} are independently (C₄ to C₃₀) alkyl and the other R^{1a}, R^{2a}, R^{3a} and R^{4a} group or groups are (C₁-C₆) alkyl or benzyl. More preferably, one or two of R^{1a}, R^{2a}, R^{3a} and R^{4a} are independently (C₆ to C₃₀) alkyl and the other R^{1a}, R^{2a}, R^{3a} and R^{4a} groups are (C₁-C₆) alkyl or benzyl groups. Optionally, the alkyl groups may comprise one or more ester (—OCO— or —COO—) and/or ether (—O—) linkages within the alkyl chain. Alkyl groups may optionally be substituted with one or more hydroxyl groups. Alkyl groups may be straight chain or branched and, for alkyl groups having 3 or more carbon atoms, cyclic. The alkyl groups may be saturated or may contain one or more carbon-carbon double bonds (eg, oleyl). Alkyl groups are optionally ethoxylated on the alkyl chain with one or more ethyleneoxy groups.

Suitable cationic surfactants for use in hair conditioners of the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, dihydrogenated tallow dimethyl ammonium chloride (eg, Arquad 2HT/75 from Akzo Nobel), cocotrimethylammonium chloride, PEG-2-oleammonium chloride and the corresponding hydroxides thereof. Further suitable cationic surfactants include those materials having the CTFA designations Quaternium-5, Quaternium-31 and Quaternium-18. Mixtures of any of the foregoing materials may also be suitable. A particularly useful cationic surfactant for use in hair conditioners of the invention cetyltrimethylammonium chloride, available commercially, for example as GENAMIN CTAC, ex Hoechst Celanese. Another particularly useful cationic surfactant for use in hair conditioners of the invention is behenyltrimethylammonium chloride, available commercially, for example as GENAMIN KDMP, ex Clariant.

Another example of a class of suitable cationic surfactants for use in the invention, either alone or together with one or more other cationic surfactants, is a combination of (i) and (ii) below:
(i) an amidoamine corresponding to the general formula (I): 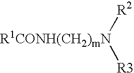
   in which R¹ is a hydrocarbyl chain having 10 or more carbon atoms,
   R² and R³ are independently selected from hydrocarbyl chains of from 1 to 10 carbon atoms, and
   m is an integer from 1 to about 10; and
(ii) an acid.

As used herein, the term hydrocarbyl chain means an alkyl or alkenyl chain.

Preferred amidoamine compounds are those corresponding to formula (I) in which
R¹ is a hydrocarbyl residue having from about 11 to about 24 carbon atoms,
R² and R³ are each independently hydrocarbyl residues, preferably alkyl groups, having from 1 to about 4 carbon atoms, and
m is an integer from 1 to about 4.

Preferably, R² and R³ are methyl or ethyl groups.

Preferably, m is 2 or 3, i.e. an ethylene or propylene group.

Preferred amidoamines useful herein include stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylmine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, and mixtures thereof.

Particularly preferred amidoamines useful herein are stearamidopropyldimethylamine, stearamidoethyldiethylamine, and mixtures thereof.

Commercially available amidoamines useful herein include: stearamidopropyldimethylamine with tradenames LEXAMINE S-13 available from Inolex (Philadelphia Pa., USA) and AMIDOAMINE MSP available from Nikko (Tokyo, Japan), stearamidoethyldiethylamine with a tradename AMIDOAMINE S available from Nikko, behenamidopropyldimethylamine with a tradename INCROMINE BB available from Croda (North Humberside, England), and various amidoamines with tradenames SCHERCODINE series available from Scher (Clifton N.J., USA).

In compositions of the invention, the level of amidoamine (i) is preferably from 0.01 to 10%, more preferably 0.1 to 7.5%, yet more preferably 0.2 to 6%, and most preferably from 0.2 to 5% by weight based on total weight of the composition.

Acid (ii) may be any organic or mineral acid which is capable of protonating the amidoamine in the hair treatment composition. Suitable acids useful herein include hydrochloric acid, acetic acid, tartaric acid, fumaric acid, lactic acid, malic acid, succinic acid, and mixtures thereof. Preferably, the acid is selected from the group consisting of acetic acid, tartaric acid, hydrochloric acid, fumaric acid, and mixtures thereof.

The primary role of the acid is to protonate the amidoamine in the hair treatment composition thus forming a tertiary amine salt (TAS) in situ in the hair treatment composition. The TAS in effect is a non-permanent quaternary ammonium or pseudo-quaternary ammonium cationic surfactant.

Suitably, the acid is included in a sufficient amount to protonate all the amidoamine present, i.e. at a level which is at least equimolar to the amount of amidoamine present in the composition.

Suitably, the acid is included in a sufficient amount to provide the hair treatment composition with a pH of between from about 2.5 to about 7.0, preferably from about 4 to about 6.5.

Typically, when an acid is present, the hair treatment compositions of the present invention comprise from about 0.1% to about 10.0% and preferably from about 0.2% to about 5.0% by weight of a suitable acid.

In conditioners of the invention, the level of cationic surfactant is preferably from 0.01 to 10%, more preferably 0.05 to 5%, most preferably 0.1 to 3% by weight of the composition.

Conditioners of the invention also incorporate a fatty alcohol. The combined use of fatty alcohols and cationic surfactants in conditioning compositions is believed to be especially advantageous, because this leads to the formation of a lamellar phase, in which the cationic surfactant is dispersed.

Representative fatty alcohols comprise from 8 to 22 carbon atoms, more preferably 16 to 22. Fatty alcohols are typically compounds containing straight chain alkyl groups.

Examples of suitable fatty alcohols include cetyl alcohol, stearyl alcohol and mixtures thereof. The use of these materials is also advantageous in that they contribute to the overall conditioning properties of compositions of the invention.

The level of fatty alcohol in conditioners of the invention is conveniently from 0.01 to 10%, preferably from 0.1 to 8%, more preferably from 0.2 to 7%, most preferably from 0.3 to 6% by weight of the composition. The weight ratio of cationic surfactant to fatty alcohol is suitably from 1:1 to 1:10, preferably from 1:1.5 to 1:8, optimally from 1:2 to 1:5. If the weight ratio of cationic surfactant to fatty alcohol is too high, this can lead to eye irritancy from the composition. If it is too low, it can make the hair feel squeaky for some consumers.

The compositions of the invention may also contain one or more further conditioning agents. As used herein, the term “conditioning agent” includes any material which is used to give a particular conditioning benefit to hair and/or skin. For example, suitable materials are those which deliver one or more benefits relating to shine, softness, combability, wet-handling, anti-static properties, protection against damage, body, volume, stylability and manageability.

Preferred conditioning agents for use in the present invention include emulsified silicones, used to impart for example wet and dry conditioning benefits to hair such as softness, smooth feel and ease of combability.

Various methods of making emulsions of particles of silicones for use in the invention are available and are well known and documented in the art.

The viscosity of the silicone itself (not the emulsion or the final washing composition) preferably ranges from 10,000 cSt (mm² sec⁻¹) to 5 million cSt (mm² sec⁻¹) at 25° C. The viscosity can be measured by means of a glass capillary viscometer as set out further in Dow Corning Corporate Test Method CTM004 Jul. 20 1970.

Suitable silicones include polydiorganosiloxanes, in particular polydimethylsiloxanes which have the CTFA designation dimethicone. An example is dimethicone fluid having a viscosity of up to 100,000 centistokes (mm² sec⁻¹) at 25° C., which is available commercially from the General Electric Company as the Viscasil series and from Dow Corning as the DC 200 series.

Aminofunctional silicones which have the CTFA designation amodimethicone, are also suitable for use in the compositions of the invention, as are polydimethyl siloxanes having hydroxyl end groups, which have the CTFA designation dimethiconol.

Also suitable are silicone gums. “Silicone gum” denotes polydiorganosiloxanes having a molecular weight of from 200,000 to 1,000,000 u and specific examples include dimethicone gums, dimethiconol gums, polydimethyl siloxane/diphenyl/methylvinylsiloxane copolymers, polydimethylsiloxane/methylvinylsiloxane copolymers and mixtures thereof. Examples include those materials described in U.S. Pat. No. 4,152,416 (Spitzer), and on General Electric Silicone Rubber product Data Sheet SE 30, SE 33, SE 54 and SE 76.

Also suitable for use in the present invention are silicone gums having a slight degree of cross-linking, as are described for example in WO 96/31188. These materials can impart body, volume and stylability to hair, as well as good wet and dry conditioning.

Preferred emulsified silicones for use in compositions of the invention have an average silicone particle diameter (D_{3,2}) in the composition of less than 100, preferably less than 30, more preferably less than 20 microns, most preferably less than 10 microns.

Particle size may be measured by means of a laser light scattering technique, using a 2600D Particle Sizer from Malvern Instruments.

Suitable silicone emulsions for use in the invention are commercially available in a pre-emulsified form. This is particularly preferred since the pre-formed emulsion can be incorporated into the washing composition by simple mixing.

Examples of suitable pre-formed emulsions include emulsions DC2-1766 and DC2-1784, and DC 1785 available from Dow Corning. These are emulsions of dimethiconol. Cross-linked silicone gums are also available in a pre-emulsified form, which is advantageous for ease of formulation. A preferred example is the material available from Dow Corning as DC X2-1787, which is an emulsion of cross-linked dimethiconol gum.

The amount of silicone incorporated into the compositions of the invention depends on the level of conditioning desired and the material used. A preferred amount is from 0.01 to about 5% by weight of the total composition although these limits are not absolute. The lower limit is determined by the minimum level to achieve conditioning and the upper limit by the maximum level to avoid making the hair and/or skin unacceptably greasy. We have found that an amount of silicone of from 0.5 to 4% by weight of the total composition, is a particularly suitable level.

A further preferred class of conditioning agents are per-alk(en)yl hydrocarbon materials, used to enhance the body, volume and stylability of hair.

EP 567 326 and EP 498 119 describe suitable peralk(en)yl hydrocarbon materials for imparting stylability and enhanced body to hair. Preferred materials are polyisobutylene materials available from Presperse, Inc. under the PERMETHYL trade name.

The amount of per-alk(en)yl hydrocarbon material incorporated into the compositions of the invention depends on the level of body and volume enhancement desired and the specific material used. A preferred amount is from 0.01 to about 10% by weight of the total composition although these limits are not absolute. The lower limit is determined by the minimum level to achieve the body and volume enhancing effect and the upper limit by the maximum level to avoid making the hair unacceptably stiff. We have found that an amount of per-alk(en)yl hydrocarbon material of from 0.5 to 2% by weight of the total composition is a particularly suitable level.

Compositions of the invention also comprise water. Typically, water is present in an amount of from 10% to 95% by weight of the composition, preferably, from 25% to 95% by weight, more preferably 30% to 95% by weight, most preferably 35% to 95% by weight.

Compositions of the invention may also optionally include a viscosity enhancer.

Examples of viscosity enhancers include:
cellulose derivatives such as methylcellulose, hydroxymethylcellulose, hydroxyethyl cellulose, hydroxypropylcellulose, and hydroxypropyl methylcellulose;
water-soluble salts of cellulose ethers such as sodium carboxymethyl cellulose and sodium carboxymethyl hydroxyethyl cellulose;
natural gums such as carrageenan, xanthan gum, gum arabic, gum tragacanth and guar gum and derivatives thereof such as hydroxypropyl guar and guar hydroxypropyl trimonium chloride;
inorganic thickeners such as colloidal magnesium aluminium silicate (Veegum), finely divided silica, natural clays such as bentonite and synthetic clays such as the synthetic hectorite available as Laponite (ex Laporte Industries Ltd);
vinyl-type polymeric thickeners such as polyvinylpyrrolidone, polyvinyl alcohol, sodium acrylate/vinyl alcohol copolymers and carboxyvinyl polymers, such as those polymers of acrylic acid cross-linked with about 0.75% to 2.0% of polyallylsucrose or polyallylpentaerythritol, obtainable under the Carbopol trademark from B.F.Goodrich.

As the viscosity enhancer, cellulose derivatives are particularly preferred, especially hydroxyethyl cellulose.

Compositions of this invention may optionally contain any other ingredient normally used in hair treatment formulations. These other ingredients may include viscosity modifiers, preservatives, colouring agents, polyols such as glycerine and polypropylene glycol, chelating agents such as EDTA, antioxidants, fragrances, and sunscreens. Each of these ingredients will be present in an amount effective to accomplish its purpose. Generally these optional ingredients are included individually at a level of up to about 5% by weight of the total composition. The method of treating hair according to the invention comprises applying to the hair a composition of the invention. The method preferably involves the application of the composition to wet hair, typically after it has been washed, normally at room temperature. Preferably, after treatment with the composition, the treated hair is rinsed with water and dried at a temperature above room temperature or allowed to dry at room temperature.

Surface Active Polymer

It is preferred if compositions according to the invention further comprise a surface active block copolymer. This is a block copolymer based on polyethyleneoxide (EO) and polypropyleneoxide (PO) blocks. Suitably, the mean molecular weight of the block copolymer is 1000 unified atomic mass units or more, preferably 2000 or more, more preferably 4000 or more, most preferably 8000 or more.

The mean molecular weight is suitably measured by determining the hydroxyl number for the polymer then transforming this into molecular weight. This corresponds to a number based mean molecular weight.

Suitable EO/PO block copolymers according to formula I have the CTFA designation Poloxamer.

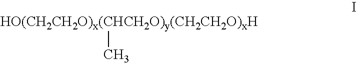

These are commercially available under the trade name “Pluronic” from BASF. Suitably, the mean value of y is from 18 to 60, preferably from 30 to 60. The mean value x is from 7 to 140 such that the ratio x/y is from 0.4 to 3.0, preferably from 1.0 to 2.7.

In formula I, the degree of polymerisation, x, is indicated as the same for each polyethyleneoxide block. For the sake of clarity, it should be explained that these degrees of polymerisation are mean values and are approximately the same rather than identical for any particular formula. This is a result of the polymerisation methods used for production of the compounds.

Another suitable block copolymer is according to formula II and has the CTFA designation “Poloxamine”.

Formula II:

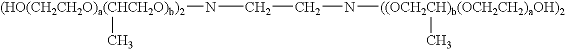

Poloxamines are commercially available from BASF under the trade name “Tetronic”. Suitably the mean value of a is 2 or more and the mean value of b is 2 or more.

Preferably, the mean value of a is 3 or more and the mean value of b is 3 or more. It is also preferred if the ratio a/b is from 0.1 to 15, more preferably from 0.5 to 6.

In formula II, the degrees of polymerisation, a and b are indicated as the same for each polyethyleneoxide and polypropylene block respectively. For the sake of clarity, it should be explained that these degrees of polymerisation are mean values and are approximately the same rather than identical for any particular formula. This is a result of the polymerisation methods used for production of the compounds.

Suitably, the level of surface active block copolymer is in the range from 0.005% to 5% by weight of the composition, preferably from 0.01% to 2%, more preferably from 0.01% to 0.7%, even more preferably from 0.01% to 0.4%.

The invention will now be illustrated with reference to the following non-limiting examples. In the examples and throughout this specification all percentages are percentages by weight unless indicated otherwise.

### EXAMPLES

Hair Fibre Modifying Agents

The following method can be employed to determine whether a substance can act as a hair fibre modifying agent that is capable of reducing the stiffness of a hair fibre.
1. 20 single Hispanic fibres were crimped with brass crimps at each end of the fibre such that the distance between the two crimps was 30 mm and were equilibrated at a fixed temperature and humidity (22° C./50% RH) for at least 24 hours.
2. A single fibre with crimps is bent and secured such that the 30 mm fibre is in the shape of a U.
3. The housing containing the fibre is attached to a load cell.
4. The load cell is operated such that the fibre is compressed against the plate of a sensitive balance at a constant load.
5. A computer collects the data from the balance, such that a force/time curve can be determined.
6. The slope of the force/time curve at the initial point of impact of the fibre with the balance is determined—this slope is used as a baseline measure.
7. The crimped fibres were soaked in the diluted treatment (4 parts water to 1 part substance), followed by rinsing by distilled water for 10 minutes.
8. The fibres were allowed to dry and then re-equilibrated for 24 hours at the same fixed temperature and humidity.
9. Steps 2) to 6) were repeated.
10. The % reduction in fibre stiffness was calculated by: 1-Slope after treatment/slope before treatment.

The materials used in the examples are as follows:

| Chemical | Trade name | Supplier |
|---|---|---|
| Behenyltrimethyl | Genamin KDMP | Clariant |
| ammonium chloride (BTAC) | | |
| Cetostearyl alcohol | Laurex | Albright &Wilson |
| Dimethiconol | DC1785 | Dow Corning |
| Quaternium 18 bentonite | Tixogel MP100 | Sud Chemie |
| Isopar L emulsion: | | |
| C11-C13 Iso-paraffin oils | Isopar L | Exxon |
| Cetyltrimethyl | | |
| ammonium chloride | Arquad 16/50 | Akzo Nobel |

### Examples 1 to 6

The following compositions were prepared. All had a pH of 5 approximately 4.5:

| | Exam- | | | | |
|---|---|---|---|---|---|
| | ple | Example | Example | Example | Example |
| Component | 1 | 2 | 3 | 4 | 5 |
| BTAC | 2.00 | 2.00 | 2.00 | 2.00 | 2.00 |
| Cetostearyl | | | | | |
| alcohol | 4.00 | 4.00 | 4.00 | 4.00 | 4.00 |
| Quaternium18 | | | | | |
| bentonite | 0.10 | 1.00 | 2.00 | 0.10 | 0.10 |
| Dimethiconol | — | — | — | 1.00 | 1.00 |
| C11-C13 | | | | | |
| Isoparaffin oil | — | — | — | — | 0.50 |
| Water/minors | to 100 | to 100 | to 100 | to 100 | to 100 |

| | Example | Control | Control | |
|---|---|---|---|---|
| Component | 6 | 1 | 2 | |
| BTAC | 2.00 | 2.00 | 2.00 | |
| Cetostearyl | | | | |
| alcohol | 4.00 | 4.00 | 4.00 | |
| Quaternium18 | | | | |
| bentonite | 0.10 | — | — | |
| Dimethiconol | 1.00 | 1.00 | 1.00 | |
| C11-C13 | | | | |
| Isoparaffin oils | — | — | 0.50 | |
| Urea | 0.50 | — | — | |
| Water/minors | to 100 | to 100 | to 100 | to 100 |

The figures in the above tables refer to % by weight of active ingredient based on total composition.

The compositions of the invention were prepared as follows. Minors such as preservative were dissolved in water at 85° C. The BTAC and cetostearyl alcohol were melted and the quaternium 18 bentonite was mixed into them. The resulting mixture was added to the solution of preservative in water at 85° C. and the whole was cooled with slow stirring to room temperature. Dimethiconol and minors were added at room temperature.

### Example 7

The friction reduction obtained on wet and dry hair was determined.

2.5 g, 150 mm (6″) European Dark Brown hair switches are used in this protocol. Individual 2.5 g, 150 mm (6″) switches (5 per product) are mounted on a block. These are held in place using clamps at either end. After fixing one end, the hair array is combed through. The array is held under tension and the second clamp is fixed in place. The ends of the switch are then cut off to leave the clamped switch ready for use. Switches are first washed with a base shampoo. Individual switches are measured using the texture analyser. They are then dried at 50° C. for 30 minutes and measured dry using the texture analyser. Conditions for measuring are set at 20° C. and 50% RH.

Each switch, after the procedure above, is treated with 0.5 ml of conditioner for 30 seconds and rinsed in running water as above for 30 seconds. The texture analyser is then used to measure the friction of the wet switch. Again, following this measurement, hair arrays are left to dry at 50° C. When dry, the switch is then measured again.

For each test, one friction loop is acquired on each array. To acquire friction data, the block, with the hair array mounted, is tested using a friction probe.

The area of each friction loop is calculated. This is taken as a measure of friction. Mean and standard deviation values of friction are recorded for each sample tested. The percentage reduction in friction is also calculated using the mean before and after measurements.

The results were as follows:

| | % friction reduction | % friction reduction |
|---|---|---|
| Composition | (wet hair) | (dry hair) |
| Control 1 | 42 | 8 |
| Control 2 | 40 | 19 |
| Example 4 | 40 | 9 |
| Example 5 | 46 | 35 |

### Example 8

The hair array volume after treatment according to the invention was determined.

Five 2 g/250 mm (10 inch) European hair switches were used for each sample to be measured. Five switches were treated at the same time by first base washing the switches and then treating with the test product.

The switches were wet with running water at ¾ litres per minute and at 35/40° C. for about 20 seconds and the excess water removed by running the thumb and forefinger along the length of the switch. 1 g of shampoo was applied along the length of the switch and agitated for 30 seconds. The switch was rinsed under the warm running water for 1 minute.

2 g of conditioner composition (according to the invention or control) was applied along the length of the five switches together and agitated for 1 minute. The switches were then rinsed with the warm running water for 1 minute. The switches were then clipped to a panel stand and detangled with a comb and then combed through once.

The switches were then hung to dry clipped to the panel stand in a humidity controlled room (22° C./20% RH) until dry.

The switches were photographed and the images analysed for volume and fluff.

The results were as follows:

| Composition | Relative Hair Array Volume |
|---|---|
| Control* | 58 |
| Example 1 | 71 |
| Example 2 | 73 |
| Example 3 | 81 |

*2% BTAC, 4% cetostearyl alcohol, balance water and minors

### Example 9

Compositions of the invention were evaluated for their conditioning effects on hair by an experienced panel of assessors. The results were as follows:

| Composition | Slippery | Smooth | Moisturised |
|---|---|---|---|
| Example 2a* | 72 | 71 | 74 |
| Control 1 | 75 | 66 | 74 |
| Example 6 | 78 | 67 | 72 |
| Example 5 | 71.5 | 71.03 | |
| Control 1 | 70.9 | 65.02 | |

*Example 2 with 1.00% dimethiconol.

### Example 10

A half head test was carried out in vivo by experienced panellists to determine the conditioning effect of the compositions. The average scores (out of 10) and number of persons preferring each product were as follows:

Average Scores:

| | Water | | | |
|---|---|---|---|---|
| Composition | Sensation | Smooth | Slippery | Manageability |
| Control 1 | 6.4 | 7.2 | 6.25 | 4.88 |
| Control 2 | 6.2 | 6.5 | 6.75 | 6.25 |
| Example 5 | 7.9 | 8.1 | 7.15 | 6.75 |

Numbers of Persons Preferring the Product:

| | Control 1 | no difference | Example 5 |
|---|---|---|---|
| Water sensation | 2 | 2 | 9 |
| Smooth | 2 | 3 | 8 |
| | Control 1 | no difference | Example 6 |
| Water sensation | 1 | — | 8 |
| Smooth | 2 | 1 | 6 |

1. A hair conditioning composition with a pH of 8 or less comprising:
(a) 0.01% to 10% by weight of one or more cationic surfactants;
(b) 0.01% to 10% by weight of one or more fatty alcohols having from 8 to 22 carbon atoms;
(c) 0.001% to 5% by weight of a hydrophobically modified clay; and
(d) water,
wherein the weight ratio of cationic surfactant to fatty alcohol is from 1:1 to 1:10.

2. A composition as claimed in claim 1, which further comprises one or more hair fibre modifying agents that are capable of reducing the stiffness of a hair fibre.

3. A composition as claimed in claim 2, wherein the hair fibre modifying agents are selected from: mineral oils; functionalised oils comprising one or more groups selected from ether, ester, keto, aldehyde, carboxyl, alcohol, diol, polyol, amino, amido, thiol and thioether, and containing 8 to 44 carbon atoms; saccharide polyesters; silicone oils; and triglyceride oils.

4. A composition as claimed in claim 3, wherein the hair fibre modifying agent is a hydrocarbon oil.

5. A composition as claimed in claim 2 wherein the hair fibre modifying agents are selected from: amino acids; urea; methyl amines and their N-oxides; mono-, di- or poly-carboxylic acids; carbohydrates; and polyols.

6. A composition as claimed in claim 5, wherein the hair fibre modifying agent is urea.

7. A composition as claimed in claim 2, wherein the one or more hair fibre modifying agents are present in the composition in an amount of from 0.01% to 2% by weight.

8. A composition as claimed in claim 1, wherein the hydrophobically modified clay is an expandable three-layer clay comprising at least 75% by weight of the clay of atoms selected from oxygen, silicon and aluminum and/or magnesium.

9. A composition as claimed in claim 1, wherein the hydrophobically modified clay is a hydrophobically treated bentonite clay.

10. A composition as claimed in claim 1, wherein the clay is hydrophobically modified by exchange into the clay of cations comprising one or more alkyl groups containing from 6 to 30 carbon atoms.

11. A composition as claimed in claim 10, wherein the cations have the formula N⁺R¹R²R³R⁴ wherein R¹, R², R³ and R⁴ are independently (C₁ to C₃₀) alkyl or benzyl and, optionally, the alkyl groups may comprise one or more ester (—OCO— or —COO—) and/or ether (—O—) linkages within the alkyl chain.

12. A composition as claimed in claim 1 which further comprises from 0.01% to 5% by weight of a silicone.

13. A composition as claimed in claim 1 which further comprises a block copolymer with a mean molecular weight of 1000 unified atomic mass units or more, comprising polyethyleneoxide and polypropyleneoxide blocks, selected from the group consisting of (i) poloxamers according to formula I: 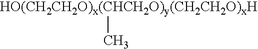
wherein the mean value of y is from 18 to 60 and the mean value x is from 7 to 140 such that the mean value of the ratio x/y is from 0.4 to 3.0.
and (ii) poloxamines according to formula II: 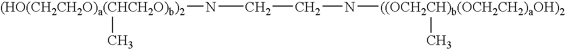
wherein the mean value of a is 2 or more and the mean value of b is 2 or more.

14. A method of treating hair which comprises applying to the hair a composition of claim 1.

15. Use of a hydrophobically modified clay for imparting conditioning benefits to hair.

16. Use as claimed in claim 15, wherein the hydrophobically modified clay is a clay which is hydrophobically modified by exchange into the clay of cations comprising one or more alkyl groups containing from 6 to 30 carbon atoms.

17. Use as claimed in claim 15 wherein the hydrophobically modified clay is present in a rinse off hair conditioning composition.

18. Use as claimed in claim 15, wherein the hydrophobically modified clay is used together with a hydrocarbon oil or urea.
